(19) United States
(12) Patent Application Publication
(10) Pub. No.: US 2005/0163725 A1
(43) Pub. Date: Jul. 28, 2005

(54) Method for administration of growth hormone via pulmonary delivery

(76) Inventors: Charles D. Blizzard, Westwood, MA (US); John J. Chipman, Carmel, IN (US); Gordon B. Cutler JR., Carmel, IN (US); Blair Jackson, South Grafton, MA (US); Lloyd Johnston, Newton, MA (US); Richard A. Lucas, London (GB); Jeffrey Mintzes, Mansey, NY (US)

Correspondence Address: ELMORE CRAIG & VANSTONE, P.C., 209 MAIN STREET, N. CHELMSFORD, MA, 01863, US

(21) Appl. No.: 10/900,992

(22) Filed: Jul. 28, 2004

Related U.S. Application Data
(63) Continuation of application No. PCT/US03/08658, filed on Mar. 19, 2003.
(60) Provisional application No. 60/366,488, filed on Mar. 20, 2002.

(51) Int. Cl. A61K 38/27; A61L 9/04; A61K 9/14

(52) U.S. Cl. 424/46; 514/2

(57) ABSTRACT

The claimed invention relates to a method of treating a human patient with growth hormone deficiency or a non-growth hormone deficiency disorder treatable with hGH, which comprises administering human growth hormone to the deep lung to said patient by a pulmonary device.

### RELATED APPLICATION

This application is a continuation of International Application No. PCT/U.S.03/08658, which designated the United States and was filed on Mar. 19, 2003, published in English, which claims the benefit of U.S. Provisional Application No. 60/366,488 Filed 20 Mar. 2002.

This application is related to U.S. application Ser. No. 10/394,401, which is also related to International Application No: PCT/U.S.03/08660, both filed on Mar. 19, 2003. This application also claims the benefit of U.S. Provisional Application No. 60/366,488, filed Mar. 20, 2002. The entire teachings of the above applications are incorporated herein by reference.

### BACKGROUND OF THE INVENTION

Human growth hormone (hGH) is a single polypeptide chain consisting of 191 amino acids. hGH therapy via subcutaneous administration has been proven to treat growth hormone deficiency in pediatric and adult patients, short stature associated with Turner syndrome, achondroplasia, Prader-Willi Syndrome, chronic renal insufficiency and children born Small for Gestational Age (SGA). There has been much interest recently in the investigation of alternative routes of delivery to injection. One such route is the systemic administration of hGH via the alveolar regions of the lung. There have been several studies involving the intratracheal administration of hGH in rats, rabbits and baboons. However, the feasibility of administering hGH to patients via the lungs with inhalers has not been demonstrated.

Thus, there is a need for a convenient, effective, and reliable method to deliver a therapeutic dose of hGH by a pulmonary device. Accordingly, it is an object herein to provide a method of treating growth hormone deficiency and non-growth hormone deficiency disorders treatable with hGH in pediatric and adult patients by intrapulmonary administration of hGH, obviating the requirement for injections or infusions.

### SUMMARY OF THE INVENTION

The present invention demonstrates that pulmonary administration of a given dosage of hGH to human patients results in clinically significant and reproducible serum levels of hGH comparable to serum levels found when dosing hGH subcutaneously.

The present invention provides a method of treating a human patient in need of hGH, for example, with growth hormone deficiency or a non-growth hormone deficiency disorder treatable with human growth hormone which comprises administering to the deep lung of said patient by a pulmonary device insert into the mouth, a pharmaceutical composition of human growth hormone of about 0.01 mg/kg administered daily to about 2.0 mg/kg administered daily.

The present invention further provides the administration of a therapeutically effective amount of hGH by a pulmonary device to adult and pediatric human patients suffering from: growth hormone deficiency; and pediatric patients with short stature due to Turner Syndrome in patients whose epiphyses are not closed; Non-Growth Hormone Deficient Short Stature (NGHDSS); Small for Gestational Age (SGA); SHOX deficiency; achondroplasia; Prader-Willi Syndrome; chronic renal insufficiency; and, any other indication of hGH.

The present invention further provides a method of treating a human patient with growth hormone deficiency or a non-growth hormone deficiency disorder treatable by a pulmonary device inserted into the mouth with a pharmaceutical composition of human growth hormone, wherein said pharmaceutical composition comprises particles, and wherein said particles are delivered from an inhalation device suitable for pulmonary administration and capable of depositing the particle in the deep lung (alveoli) of the patient.

In a preferred embodiment of the present invention, the particle comprises human growth hormone and a buffer. For example, the particle may consist of 93.5% human growth hormone and 6.5% sodium phosphate by weight.

The present invention further provides the use of human growth hormone in the manufacture of a medicament for the treatment of growth hormone deficiency or a non-growth hormone deficiency disorder by a pulmonary device at a dose of about 0.01 mg/kg administered daily to about 2.0 mg/kg administered daily.

The present invention further provides an article of manufacture comprising packaging material and a pharmaceutical agent contained within said packaging material, wherein said pharmaceutical agent is effective for treating a patient with growth hormone deficiency or a non-growth hormone deficiency disorder treatable with human growth hormone and wherein said packaging material comprises a label which indicates that said pharmaceutical agent comprises human growth hormone administered by a pulmonary device at a dose of about 0.01 mg/kg administered daily to about 2.0 mg/kg administered daily or alternatively 0.07 mg/kg administered weekly to about 14 mg/kg administered weekly, divided into equal doses given either on 3 alternate days or 6 times per week.

The present invention further provides a pharmaceutical composition in a unit dosage form comprising a dry powder suitable for pulmonary administration by a patient, said unit dosage form comprising human growth hormone and a buffer.

### DETAILED DESCRIPTION OF THE INVENTION

This invention encompasses methods for treating patients in need of human growth hormone therapy which includes but is not limited to: the use in long-term treatment of pediatric patients who have growth failure due to an inadequate secretion of normal endogenous growth hormone; the treatment of short stature associated with Turner Syndrome in patients whose epiphyses are not closed; for the treatment of Small for Gestational Age (SGA); the treatment of short stature homeobox gene defects (SHOX deficiency); achondroplasia; Prader-Willi Syndrome, chronic renal insufficiency associated with short stature in pediatric patients; patients suffering from AIDS wasting; for replacement of endogenous growth hormone in adults with growth hormone deficiency; and for any other indication of hGH. Aspects of the present invention include pharmaceutical compositions of human growth hormone and strategies of administrating the same.

The term “growth hormone” refers to (1) growth hormone itself of whatever species, for example, human, bovine, or porcine, although the present invention is particularly applicable to human growth hormone (hGH); (2) precursors to growth hormone, such as reduced (—SH) growth hormone and S-protected growth hormone, for example, growth hormone S-sulfonate; (3) variants of growth hormone or its precursors, for example, structures which have been modified to lengthen and/or shorten the growth hormone amino acid sequence, for example, the 20K variant of growth hormone, methionyl growth hormone, and the like; (4) analogs of growth hormone or its precursors, for example, a molecule having one or more amino acid substitutions, deletions, inversions, or additions compared with growth hormone; and (5) derivatives of growth hormone or its precursors, for example, a molecule having the amino acid sequence of growth hormone or growth hormone analog, but additionally having chemical modification of one or more of its amino acid side groups, alpha-carbon atoms, terminal amino groups, or terminal carboxylic acid groups.

Pharmaceutical Compositions:

The pharmaceutical composition of hGH utilized in the present invention refers to a powder or suspension that comprises particles of hGH which can be efficaciously administered by a pulmonary device inserted into the mouth and capable of delivering said particles of hGH to the deep lung of a patient.

The nature and quantity of the pharmaceutical composition and the duration of administration of a single dose depend on the type of inhalation device employed. For some aerosol delivery systems, such as nebulizers, the frequency of administration and length of time for which the system is activated will depend on the concentration of hGH in the powders of the aerosol. For example, shorter periods of administration can be used at higher concentrations of the hGH powders in the nebulizer solution. Devices such as metered dose inhalers can produce higher aerosol concentrations, and can be operated for shorter periods to deliver the desired amount of the powders. Devices such as dry powder inhalers deliver active agent until a given charge of agent is expelled from the device. In this type of inhaler, the quantity of therapeutic protein particles in a given quantity of the powder determines the dose delivered in a single administration.

The pharmaceutical composition of hGH may contain a buffer which could include phosphate such as sodium phosphate monohydrate and dibasic sodium phosphate, TRIS, maleate, acetate such as sodium acetate, citrate such as sodium citrate, sodium tartrate, or amino acids such as glycine, glycylglycine, histidine, lysine, or arginine. Other pharmaceutically acceptable buffers are known in the art. Preferably, the buffer is selected from the group consisting of sodium phosphate, TRIS, maleate, and glycine. Even more preferably the buffer is sodium phosphate. Preferably, the sodium phosphate in the particles is between about 3% and about 20%. More preferably, the percent is between about 3.5% and about 15%. Even more preferably, the percent is between about 4% and about 10%. Most preferably, the percent is between about 5.5% and 7.5%. An exemplary amount of sodium phosphate in the particles is 6.5%.

The pharmaceutical composition of hGH may optionally encompass an additive, such as a bulking agent, carrier, or excipient. Additives can be included in the dry powder to dilute the powder as required for delivery from the particular powder inhaler, to facilitate processing of the pharmaceutical composition, to provide advantageous powder properties to the pharmaceutical composition, to facilitate dispersion of the powder from the inhalation device, to stabilize the pharmaceutical composition (e.g., antioxidants or buffers), to provide taste to the pharmaceutical composition, or the like. Advantageously, the additive does not adversely affect the patient's airways. Typical additives include mono-, di-, and polysaccharides; sugar alcohols and other polyols, such as, for example, lactose, glucose, raffinose, melezitose, lactitol, maltitol, trehalose, sucrose, mannitol, starch, or combinations thereof; surfactants, such as sorbitols, diphosphatidyl choline, or lecithin; amino acids, such as arginine, glycine, and leucine; or the like. Typically an additive, such as a bulking agent, is present in an amount effective for a purpose described above, often at about 50% to about 90% by weight of the pharmaceutical composition.

The pharmaceutical composition of hGH may optionally encompass one or more additional components. Generally, the amount of the additional component(s) is less than 50 weight percent, preferably less than 30 weight percent and most preferably less than 20 weight percent. Preferred are particles that include, in addition to the growth hormone and buffer salt(s), one or more phospholipids. Specific examples of phospholipids include but are not limited to phosphatidylcholines dipalmitoyl phosphatidylcholine (DPPC), dipalmitoyl phosphatidylethanolamine (DPPE), distearoyl phosphatidylcholine (DSPC), dipalmitoyl phosphatidyl glycerol (DPPG) or any combination thereof.

Indications:

An aspect of the present invention relates to a method of treating adult and pediatric Growth Hormone Deficient (GHD) patients with hGH by a pulmonary device. Pulmonary efficacy (height velocity) is at least equivalent to subcutaneous therapy in pediatric patients. This treatment results in a mean height velocity in GHD pediatric patients that is comparable to subcutaneous injection over 12 months (e.g. statistically significant by non-inferiority to subcutaneous injection; 95% confidence interval for pulmonary >66.7% of mean height velocity of subcutaneous). Surprisingly, no clinically and statistically significant increase in growth-inhibiting antibody formation, i.e. statistically significant with evidence of growth inhibition, compared to subcutaneous injection in long term studies (>12 months) is observed.

Another aspect of the present invention relates to a method of treatment comprising the administration of a therapeutically effective amount of hGH by a pulmonary device to patients suffering from non-growth hormone deficiency disorders treatable with hGH which include: Turner Syndrome in patients whose epiphyses are not closed; Non-Growth Hormone Deficient Short Stature (NGHDSS); Small for Gestational Age (SGA); SHOX deficiency; achondroplasia; Prader-Willi Syndrome; chronic renal insufficiency; patients suffering from AIDS wasting; and, for any other indication of hGH.

Dosing:

The hGH utilized in the methods of the present invention is dosed based on the medical indication and body weight of the patient or total daily dose in adult patients with growth hormone deficiency. According to the present invention, hGH is administered by pulmonary delivery to achieve absorption in the lungs relative to subcutaneous administration of hGH. Efficacious serum levels of hGH are achieved by subcutaneous dosing regimens ranging from about 0.02 mg/kg/week up to about 0.7 mg/kg/week divided into daily doses. Therefore, a single daily dose would range from about 0.003 mg/kg/day to about 0.1 mg/kg/day. Consequently, in order to achieve the efficacious serum levels after pulmonary delivery, it has been determined that the preferable dose needs to be about 5 fold to about 20 fold above the subcutaneous dose (about 0.1 mg/kg/week to about 14 mg/kg/week, and the daily dosing regimens range from about 0.01 mg/kg/day to about 2 mg/kg/day). More preferably, the pulmonary dose needs to be about 10 fold to about 18 fold above the subcutaneous dose (about 0.2 mg/kg/week to about 12.6 mg/kg/week, and the daily dosing regimens range from about 0.03 mg/kg/day to about 1.8 mg/kg/day). Most preferably, the pulmonary dose needs to be about 14-16 fold above the subcutaneous dose (about 0.3 mg/kg/week to about 11.2 mg/kg/week, and the daily dosing regimens range from about 0.04 mg/kg/day to about 1.6 mg/kg/day).

For example, the current recommended dosage for growth hormone deficient pediatric patients is about 0.18 mg/kg/week to about 0.3 mg/kg/week, divided into equal doses given either on 3 alternate days, 6 times per week, or daily. The treatment can be continued until final height or closure of the epiphyses, often 4-7 years duration. A comparable dose for pulmonary administration is about 0.9 mg/kg/week to about 1.5 mg/kg/week to a maximum of about 3.6 mg/kg/week to about 6.0 mg/kg/week, divided into equal doses given either on 3 alternate days, 6 times per week, or daily.

The current recommended therapy for Turner Syndrome is a weekly dosage of up to 0.375 mg/kg of body weight administered by subcutaneous injection divided into equal dose given either daily or on 3 alternate days. Treatment is to final height, 4-6 year duration. A comparable therapeutic dose for pulmonary administration would be from about 1.875 mg/kg of body weight to about 7.5 mg/kg of body weight divided into equal doses given either daily or on 3 alternate days.

In addition, the current recommended dosage for growth hormone deficient adult patients may begin at 0.003 mg/kg/day given as a daily subcutaneous injection and may be increased accordingly to individual patient requirements to a maximum of 0.0125 mg/kg/day. Duration of therapy could be for life. A comparable therapeutic dose for pulmonary administration would be from about 0.015 mg/kg/day to about 0.12 mg/kg/day to a maximum of 0.0625 mg/kg/day to about 0.25 mg/kg/day.

Thus, the pulmonary administration of hGH of the present invention will provide similar dosage and dose flexibility as subcutaneous injection of hGH for Turner Syndrome and growth hormone deficient adult and pediatric patients as well as Non-Growth Hormone Deficient Short Stature (NGHDSS), Small for Gestational Age (SGA), SHOX deficiency, achondroplasia, Prader-Willi Syndrome, patients suffering from AIDS wasting; chronic renal insufficiency associated with short stature in pediatric patients; and any other indication for hGH therapy.

Devices:

There are many devices known in the art that are useful for administering the particles comprising hGH by inhalation into the deep lungs of a patient in need of such treatment. Included among the devices that may be used to administer the powder according to the present invention include metered dose inhalers, liquid nebulizers, dry powder inhalers, sprayers, thermal vaporizers, and the like, but does not include an intratracheal device or an intranasal device or delivery route. Preferably, the inhalation device is easy to use, small enough to carry conveniently, capable of providing multiple doses, and durable. Examples of such devices include those described in U.S. patent application Ser. No. 10/101,563 entitled “A Method and Apparatus for Producing Dry Particles”, herein incorporated by reference. Other possible devices include the AERxφ pulmonary drug delivery system being developed by Aradigm Corporation, the dry powder and delivery devices being developed by Inhale Therapeutic Systems, Inc., and the Spiros® dry powder inhaler system being developed by Dura Pharmaceuticals, Inc., electrohydrodynamic aerosolizers being developed at Battelle and devices that use piezoelectric ultrasonic particle generators, such as the AeroDose™ Inhalers developed by AeroGen, Inc. Some specific examples of commercially available inhalation devices suitable for the practice of this invention are Turbuhaler® (Astra), Rotahaler® (Glaxo), Diskus® ((Glaxo), the Ultravent® nebulizer (Mallinckrodt), the Acorn II nebulizer (Marquest Medical Products), the Ventolin® metered dose inhaler (Glaxo), the Spinhaler® powder inhaler (Fisons).

The particular device chosen for the present invention is not critical. However, in order to achieve the required dosing regimens, the device will need to be able to deliver a dose in the range of about 2 mg to about 130 mg of hGH. In another embodiment, the device will need to be able to deliver a dose in the range of about 15 mg to about 80 mg. In another embodiment, the device will need to be able to deliver a dose in the range of about 50 mg to about 80 mg. In another embodiment, the device will need to be able to deliver a dose in the range of about 50 mg to about 65 mg.

An alternative means of determining the amount of hGH that will need to be delivered for an efficacious dose to a patient is the relative bioavailability of the pulmonary dose as compared to blood levels following a subcutaneous dose. In general, bioavailability can be estimated by performing area under the curve (AUC) calculations.

The present invention has determined that the relative bioavailability of a pulmonary dose in humans is from about 5% to about 10% of the amount of hGH that is in the capsule prior to delivery. In another embodiment, the relative bioavailability of a pulmonary dose in humans is from about 6% to about 8% relative to blood levels following a subcutaneous dose. In other words, over the time course of the human clinical study described in Pharmaceutical Study 1, relative bioavailability of hGH administered by a pulmonary device is approximately 6 to 8% relative to the amount of hGH that is in the capsule prior to pulmonary administration relative to blood levels following a subcutaneous dose.

Process

Methods for preparing the hGH, hGH analogs, or hGH derivatives useful in the present invention are well-known in the art and are easily within the grasp of ordinarily skilled protein chemists or biochemists. The amino acid portion of the active compound used in the present invention, or a precursor thereto, can be made either by solid-phase synthetic chemistry, purification of hGH molecules from natural sources, or recombinant DNA technology. Routine synthetic organic techniques enable the alkylation and acylation of the hGH derivatives.

The methods of the present invention include the use of hGH particles useful for delivery of hGH to the pulmonary system, in particular to the deep lung. In one example, the particles preferably are in the form of a dry powder and are characterized by a fine particle fraction (FPF), geometric and aerodynamic dimensions and by other properties, as further described in U.S. Provisional Patent Application No. 60/366,488, filed concurrently herewith.

Article of Manufacture

The invention also contemplates an article of manufacture that is a labeled container for providing human growth hormone. An article of manufacture comprises packaging material and a pharmaceutical agent contained within the packaging material.

The pharmaceutical agent in an article of manufacture is human growth hormone of the present invention, formulated into a pharmaceutically acceptable form as described herein according the disclosed indications. The article of manufacture contains an amount of pharmaceutical agent sufficient for use in treating a condition indicated herein, either in unit or multiple dosages.

The packaging material comprises a label that indicates the use of the pharmaceutical agent contained therein, e.g., treating a subject with growth hormone deficiency or a non-growth hormone deficiency disorder, and like conditions disclosed herein. The label can further include instructions for use and related information as may be required for marketing. The packaging material can include container(s) for storage of the pharmaceutical agent.

As used herein, the term packaging material refers to a material such as glass, plastic, paper, foil, and the like capable of holding within fixed means a pharmaceutical agent. Thus, for example, the packaging material can be plastic or glass vials, laminated envelopes and the like containers used to contain a pharmaceutical composition including the pharmaceutical agent. In preferred embodiments, the packaging material includes a label that is a tangible expression describing the contents of the article of manufacture and the use of the pharmaceutical agent contained therein.

An embodiment of the present invention is packaging material comprising a blister package (peel-back blister) wherein said blister package contains seven capsules, each capsule containing a specific amount of the pharmaceutical composition of hGH of the present invention. Preferably, the capsule(s) contain a unit dosage of 3.0 mg, 4.8 mg, 6.0 mg, 9.0 mg, 12.0 mg, 15.1 mg, or 21.1 mg of the pharmaceutical composition of hGH of the present invention. The total dosage administered to the patient is based on the patient's body weight (e.g. mg/kg) as recommended by a physician. Any combination of the unit dosage capsules to achieve the necessary total dosage is appropriate. The frequency of administration will depend on the indication and may be daily, six days a week, five days per week, four days per week, three days per week, two days per week or one day per week. It is also contemplated that the daily dose could be divided and administered as two or more portions but not to exceed the total recommended daily dosage. Whatever the dosing frequency, the total dose administered is based on mg/kg/week, preferably divided into equal doses.

Preparation of a Pharmaceutical Composition

93.5 wt % hGH/6.5 wt % Sodium Phosphate

Particles containing hGH and sodium phosphate monohydrate were prepared as follows. The aqueous solution was prepared by preparing a bulk sodium phosphate solution at 100 mM at pH 7.4 and a bulk ammonium bicarbonate solution at 50 g/L. Fifty-two ml of 100 mM sodium phosphate buffer at pH 7.4 was added to 268 ml of water for irrigation. To this was added 200 ml of the 50 g/L ammonium bicarbonate solution and 200 ml of ethanol. The resulting solution was combined in a static mixer with 280 mL of bulk hGH at 40 g/L in 1.7 mM sodium phosphate buffer at pH 7.4. Solute concentration in the combined solution was 12 g/L. The combined solution was spray dried under the following process conditions:
Inlet temperature ˜74° C.
Outlet temperature from the drying drum ˜40° C.
Nitrogen drying gas=110 kg/hr
Nitrogen atomization gas=64 g/min
2 Fluid internal mixing nozzle atomizer
Nitrogen atomization pressure ˜90 psi
Liquid feed rate=25 ml/min
Liquid feed temperature ˜22° C.
Pressure in drying chamber =−2.0 in water

The resulting particles had a FPF(5.6) of 75%, and a FPF(3.4) of 70%, both measured using a 2-stage ACI (Anderson Cascade Impactor). The volume mean geometric diameter was 8 μm at 1.0 bar. The resulting particles had a soluble dimer fraction of 1.2% and a readily extractable hGH fraction of 97.5%.

The combination solution flowing out of the static mixer was fed into a two-fluid nozzle atomizer. The contact between the atomized droplets from the atomizer and the heated nitrogen caused the liquid to evaporate from the droplets, resulting in dry porous particles. The resulting gas-solid stream was fed to a bag filter that retained the resulting dry particles, and allowed the hot gas stream containing the drying gas (nitrogen), evaporated water, and ethanol to pass. The dry particles were collected into a product collection vessel.

In order to obtain dry particles of particular physical and chemical characteristics, in vitro characterization tests can be carried out on the finished dry particles, and the process parameters adjusted accordingly, as described, for example, in U.S. patent application Ser. No. 10/101,563. Particles containing 93.5% hGH and 6.5% sodium phosphate produced using this method had a VMGD of 8.4 μm, FPF(5.6) of 89% to 93%, readily extractable hGH fraction of 95.5%, and a soluble dimer fraction of 3%. In this manner, the desired aerodynamic diameter, geometric diameter, and particle density could be obtained for these particles in real-time, during the production process.

Study for Growth Hormone Inhalation Powder Kit

Twelve individuals were chosen for the clinical trials of the hGH Inhalation Powder Kit. Each individual was given an inhaler and inhaled the hGH formulation as follows.

Preparation

The mouthpiece was removed from the inhaler body to allow access to the capsule chamber. The number of growth hormone capsules that are required for the dose were removed from the blister package. The hGH capsules were at room temperature for at least one hour but not more than three hours. One growth hormone capsule was inserted into the capsule chamber. The mouthpiece was reattached onto inhaler body by pressing both pieces firmly together until a snap is heard and the motion stops. This action punctures the capsule, making it ready to use.

Administration Procedure

Before beginning, the subject needed to ensure that the mouth was clear of any potential obstructions. The individuals were instructed to sit upright, relax and breathe normally for at least five breaths, then remove the inhaler cap. The individuals were instructed to hold the inhaler away from their mouths, and exhale as much as possible without becoming uncomfortable, and without forcing their breath out. They inserted mouthpiece into their mouths, making sure the inhaler was held straight out from the mouth and horizontal. They took a deep breath through their mouths—until their lungs were full—removed the mouthpiece and held their breath for five seconds, before letting it out normally. This administration procedure constitutes a single, breath actuated step.

Capsule Inspection and Disposal

The mouthpiece was removed from the inhaler body, and the capsule was removed from the chamber. The capsule was inspected to make sure the dose was administered. Generally, the capsule had a light dusting of white powder on the inside and two (2) holes on the bottom. If more than a light dusting of powder remained in the capsule, the capsule was reinserted back into the capsule chamber and the above process was repeated until all the powder (except the normal dusting) was inhaled. When reinserting the capsule, the operators were asked to make sure the end of the capsule with two (2) holes was placed into the chamber first. If more than one capsule was required for the total dose, the above process was repeated with the remaining capsule(s).

Storing the Kit

Used capsules were discarded in the trash. The remaining contents were returned to the case. The case with the remaining capsules was stored in the refrigerator at the recommended storage conditions (2° C./36° F.-8° C./46° F.). After the last dose was delivered with an inhaler, the inhaler was discarded in the trash.

Safety Results

Subjects were assessed for cough, gagging and abnormal taste after pulmonary dosing. Vital signs and pulmonary function were measured up to 12 hours after dosing. Subjects were monitored for clinically significant changes. Adverse Events (ADEs) were recorded.

Pharmacokinetics Study 1:

This was a randomized, 3-period crossover study. Subjects received a maximum of approximately 19.5 mg of inhaled hGH per capsule (of which 16.7 mg is hGH monomer) and subcutaneous Humatrope® (4 mg) administered as described in the above. Twelve healthy male subjects, aged between 21 and 55 years, were enrolled and studied in each group. All subjects were required to consume a carbohydrate-rich breakfast within 30 minutes prior to dosing, on each occasion, in an attempt to reduce endogenous hGH secretion.

Two study groups were utilized: Group 1, using a pulmonary formulation with lipid, designated F2 (80% hGH, 14% DPPC and 6% sodium phosphate) and Group 2, using a pulmonary formulation without lipid, designated F3 (93% hGH and 7% sodium phosphate). Subjects received single doses of each study drug in accordance with the randomization schedule, beginning on Day 1. Each dose was separated by a washout period of at least 48 hours, such that dosing occurred for example on Days 1, 3, and 5. The study was subject blind to the pulmonary formulations during the first 2 study periods, but not during the third study period, when all subjects received subcutaneous Humatrope®.

Relative bioavailability to subcutaneous administration was approximately 6-7% (F2) and 7-8% (F3) respectively. Inhaled doses of F2 (74 mg) and F3 (78.4 mg) produce similar peak hGH concentrations and systemic exposure to subcutaneous 4 mg. Mean inspiratory flow rate was 0.84 L/sec (range 0.64 to 1.06 L/sec).

The subjects were assessed for cough, gagging and abnormal taste after pulmonary dosing. Their vital signs and pulmonary function measured up to 12 hours after dosing. There were no clinically significant changes. Data on Adverse Events (ADEs) was collected. 13 ADEs reported by ten (10) subjects, principally headache five (5), nausea one (1), and postural dizziness two (2). No coughing or issues with taste were reported.

Pharmacokinetics Study 2:

This was a single center, randomized, multiple dose, two-period crossover study. Each subject was randomized to receive either placebo in both study periods or one of three inhaled hGH doses (93.5% hGH and 6.5% sodium phosphate) during one study period, and the corresponding dose of subcutaneous Humatrope® in the alternate study period. In addition, subjects randomized to receive active study drug, were administered placebo (corresponding to the active medication they were to receive on dosing days) for 2 days. Study drug was administered as described above, once-daily for 5 days during each study period, and there was a washout period of at least 14 days between study periods. A maximum of 24 healthy male subjects, aged between 21 and 55 years, were enrolled, and studied in three groups of 8. All subjects were required to consume a carbohydrate-rich breakfast, within 30 minutes prior to dosing on pharmacokinetic blood sampling days (Days 1, 3, 6 and 7), to aid suppression of endogenous growth hormone. All subjects had screening assessments prior to study entry. For each study period, subjects were admitted to the Unit at a pre-defined time on the day before dosing commenced (Day-1). They remained resident in the Unit for the entire study period until discharge, approximately 24 hours after administration of the final dose. All subjects enrolled had a post-study examination. Group 2 commenced dosing after Group 1 completed Study Period 1, and the pulmonary function data was assessed. Also, Group 3 commenced dosing after Group 2 completed Study Period 1. Safety assessments and blood sampling for pharmacokinetic and pharmacodynamic parameters were performed pre-dose and up to 24 hours following the last dose.

The results of Pharmacokinetics Study 2 are shown in Table 1.

TABLE 1

| PK | Route of | Dose | | | | |
|---|---|---|---|---|---|---|
| Parameter | Admin. | (mg) | N | Mean | CV(%) | Median |
| AUC | Inhaled | 16.7 | 18 | 12.7 | 65.67 | 12.83 |
| (ng/ml/h) | hGH | 50.1 | 11 | 53.09 | 58 | 51.23 |
| | | 83.5 | 15 | 158.34 | 70.19 | 205.96 |
| | Subcut. | 1.0 | 18 | 11.05 | 50.37 | 12.66 |
| | HGH | 3.0 | 12 | 69.16 | 50.31 | 78.06 |
| | | 5.0 | 15 | 95.46 | 38.04 | 90.24 |
| AUC5 | Inhaled | 16.7 | 18 | 9.56 | 63.05 | 8.7 |
| (ng/ml/h) | hGH | 50.1 | 11 | 28.69 | 54.44 | 31.33 |
| | | 83.5 | 15 | 74.11 | 66.99 | 78.43 |
| | Subcut. | 1.0 | 18 | 8.16 | 49.73 | 9.24 |
| | HGH | 3.0 | 12 | 38.29 | 91.88 | 49.29 |
| | | 5.0 | 15 | 50.07 | 59.35 | 44.8 |
| Cmax | Inhaled | 16.7 | 18 | 2.94 | 52.82 | 2.95 |
| (ng/ml) | hGH | 50.1 | 11 | 8.24 | 49.55 | 8.58 |
| | | 83.5 | 15 | 20.90 | 66.43 | 22.06 |
| | Subcut. | 1.0 | 18 | 2.85 | 49.27 | 2.87 |
| | hGH | 3.0 | 12 | 12.11 | 75.71 | 14.02 |
| | | 5.0 | 15 | 14.17 | 57.4 | 11.74 |

AUC: area under curve from time of administration to last measurable concentration
AUC5: area under curve from time of administration to 5 hours post dosing
Cmax: maximum observed hGH serum concentration

The above data demonstrates that a given dosage of hGH can be administered pulmonarily to human patients resulting in clinically significant and reproducible serum levels of hGH comparable to serum levels found when dosing hGH subcutaneously. Furthermore, the data indicate that in order to obtain a comparable serum concentration level of hGH after pulmonary administration relative to that of a subcutaneous dose, it is necessary to administer approximately 16-fold more by pulmonary administration compared to the subcutaneous dose.

While this invention has been particularly shown and described with references to preferred embodiments thereof, it will be understood by those skilled in the art that various changes in form and details may be made therein without departing from the scope of the invention encompassed by the appended claims.

1. A method of treating a human patient with growth hormone deficiency or a non-growth hormone deficiency disorder treatable with human growth hormone which comprises administering to the deep lung of said patient by a pulmonary device inserted into the month, a pharmaceutical composition of human growth hormone of about 0.01 mg/kg administered daily to about 2.0 mg/kg administered daily.

2. The method according to claim 1, wherein said non-growth hormone deficiency disorder is selected from the group consisting of Turner Syndrome, Small for Gestational Age (SGA), SHOX deficiency, achondroplasia, chronic renal insufficiency, Prader-Willi Syndrome, or Non-Growth Hormone Deficient Short Stature.

3. The method according to claim 1, wherein said pharmaceutical composition of the human growth hormone is about 0.03 mg/kg administered daily to about 1.8 mg/kg administered daily.

4. The method according to claim 3, wherein said pharmaceutical composition of the human growth hormone is about 0.04 mg/kg administered daily to about 1.6 mg/kg administered daily.

5. The method of claim 4, wherein the relative bioavailability of human growth hormone administered by a pulmonary device is about 6% to about 8% relative to blood levels following a comparable subcutaneous dose.

6. The method according to claim 1, wherein said pharmaceutical composition comprises particles, wherein said particles are delivered from said pulmonary device.

7. The method of claim 6, wherein the device is selected from the group consisting of a nebulizer, a metered-dose inhaler, and a dry powder inhaler.

8. The method of claim 7, wherein the device is a dry powder inhaler.

9. The method of claim 6, wherein the particle further comprises a buffer selected from the group consisting of sodium phosphate, TRIS, maleate, and glycine.

10. The method of claim 9, wherein said buffer is sodium phosphate.

11. The method according to claim 6, wherein said particle consists of about 93.5% human growth hormone and about 6.5% sodium phosphate.

12. The use of human growth hormone in the manufacture of a medicament for the treatment of growth hormone deficiency or a non-growth hormone deficiency disorder by administration with a pulmonary device at a dose of about 0.01 mg/kg administered daily to about 2.0 mg/kg administered daily.

13. An article of manufacture comprising packaging material and a pharmaceutical agent contained within said packaging material, wherein said pharmaceutical agent is effective for treating a patient with growth hormone deficiency or a non-growth hormone deficiency disorder treatable with human growth hormone and wherein said packaging material comprises a label which indicates that said pharmaceutical agent comprises human growth hormone administered by a pulmonary device at a dose of about 0.01 mg/kg administered daily to about 2.0 mg/kg administered daily or alternatively at a dose of about 0.07 mg/kg administered weekly to about 14 mg/kg administered weekly divided into equal doses given either on 3 alternate days or 6 times per week.

14. The article of manufacture of claim 13, wherein said packaging material comprises a blister package wherein said blister package contains seven capsules, each capsule containing a unit dosage of the pharmaceutical composition of hGH.

15. The article of manufacture of claim 14, wherein said non-growth hormone deficiency disorder is selected from the group consisting of Turner Syndrome, Small for Gestational Age (SGA), SHOX deficiency, achondroplasia, chronic renal insufficiency, Prader-Willi Syndrome, or Non-Growth Hormone Deficient Short Stature.

16. A pharmaceutical composition in a unit dosage form comprising a dry powder suitable for pulmonary administration by a patient to the deep lung, said unit dosage form comprising:
a) human growth hormone; and
b) a buffer.

17. The pharmaceutical composition according to claim 16, wherein said buffer is selected from the group consisting of sodium phosphate, TRIS, maleate, and glycine.

18. The pharmaceutical composition according to claim 16, wherein said unit dosage form is a capsule.

19. The unit dosage form according to claim 18, wherein said capsule contains a unit dosage of 3.0 mg of said pharmaceutical composition.

20. The unit dosage form according to claim 18, wherein said capsule contains a unit dosage of 4.8 mg of said pharmaceutical composition.

21. The unit dosage form according to claim 18, wherein said capsule contains a unit dosage of 6.0 mg of said pharmaceutical composition.

22. The unit dosage form according to claim 18, wherein said capsule contains a unit dosage of 9.0 mg of said pharmaceutical composition.

23. The unit dosage form according to claim 18, wherein said capsule contains a unit dosage of 12.0 mg of said pharmaceutical composition.

24. The unit dosage form according to claim 18, wherein said capsule contains a unit dosage of 15.1 mg of said pharmaceutical composition.

25. The unit dosage form according to claim 18, wherein said capsule contains a unit dosage of 21.1 mg of said pharmaceutical composition.

26. A method of treating a human pediatric patient with growth hormone deficiency which comprises administering to the deep lung of said patient by a pulmonary device, a pharmaceutical composition of human growth hormone of about 0.9 mg/kg/week to about 1.5 mg/kg/week to a maximum of about 3.6 mg/kg/week to about 6.0 mg/kg/week.

27. The method according to claim 26, wherein said pharmaceutical composition is divided into equal doses given on 3 alternate days, 6 times per week, or daily.

28. The method according to claim 26, wherein said treatment is continued until final height is attained or closure of the epiphyses.

29. A method of treating a human patient with Turner Syndrome which comprises administering to the deep lung of said patient by a pulmonary device, a pharmaceutical composition of human growth hormone of about 1.9 mg/kg to about 7.5 mg/kg divided into equal doses given either daily or on 3 alternate days.

30. The method according to claim 29, wherein said treatment is continued until final height is attained.

31. A method of treating an adult human patient with growth hormone deficiency which comprises administering to the deep lung of said patient by a pulmonary device inserted into the month, a pharmaceutical composition of human growth hormone of about 0.015 mg/kg/day to about 0.12 mg/kg/day to a maximum of 0.06 mg/kg/day to about 0.25 mg/kg/day.
